(19) United States
(12) Patent Application Publication
(10) Pub. No.: US 2018/0117114 A1
(43) Pub. Date: May 3, 2018

(54) PEPTIDES THAT MODULATE THE EFFECT OF THE CRMP: NEUROFIBROMIN COMPLEX ON SYNAPTIC TRANSMISSION

(71) Applicant: Indiana University Research and Technology Coporation, Indianapolis, IN (US)

(72) Inventor: Rajesh Khanna, Tucson, AZ (US)

(73) Assignee: Indiana University Research and Technology Coporation, Indianapolis, IN (US)

(21) Appl. No.: 15/560,881

(22) PCT Filed: Mar. 25, 2016
(86) PCT No.: PCT/US16/24266

§ 371 Date: Sep. 22, 2017

Related U.S. Application Data
(60) Provisional application No. 62/138,203, filed on Mar. 25, 2015.

(51) Int. Cl. A61K 38/10; A61K 47/64; C07K 14/705; A61K 9/00; A61P 25/00

(52) U.S. Cl. CPC A61K 38/10; A61K 47/645; C07K 2319/10; A61K 9/0019; A61P 25/00; C07K 14/70571

(57) ABSTRACT

The present disclosure relates generally to materials and methods for studying and treating neurofibromatosis type 1 (NF1) by modulating the interaction of CRMP-2 and neurofibromin. Some embodiments relates to newly synthesized polypeptides or a pharmaceutically acceptable salt thereof. Other embodiments relate to methods of treating a patient by providing at least one polypeptide. Another embodiment relates to a kit comprising at least one polypeptide.

### PRIORITY CLAIM

This application claims the benefit of U.S. Provisional Patent Application No. 62/138,203 filed on Mar. 25, 2015, the disclosure of which is incorporated herein by reference in its entirety.

### FIELD OF THE INVENTION

The present disclosure relates generally to materials and methods for studying and treating neurofibromatosis type 1 (NF1) by modulating the interaction of CRMP-2 and neurofibromin.

### BACKGROUND AND SUMMARY

Neurofibromatosis type 1 (NF1) is a common genetic disorder, affecting about 1 in 3,000 people throughout the world. NF1 is characterized by developmental changes in the nervous system, skin, bones, and other tissues. Its most characteristic features are multiple benign, soft tumors called neurofibromas and patches of skin pigmentation called café-au-lait spots. NF1 also affects nerves throughout the body, including those in the brain. About half of the people with NF1 suffer from learning and memory deficits. These deficits are usually identified around 3-16 years of age but can persist throughout life and may affect the ability of a person to obtain an education and meaningful employment. In individuals with NF1, the lack of the protein neurofibromin allows for unchecked activation of signalling pathways affecting neurons. Given the devastating effects of this disorder and the dearth of effective treatments for this condition, there is an on-going need for new therapies to treat this condition. One object of the present disclosure herein is to help to treat this disease.

Neurofibromatosis type I (NF1) is a common autosomal dominant disease characterized by the formation of multiple benign and malignant tumors. People with NF1 often have learning disabilities and other cognitive symptoms. The mechanisms by which mutations of the neurofibromin gene (NF1) cause these deficits are not known. Learning and memory deficits also are observed in mice with a heterozygous mutation of the Nfl gene (Nfl^{+/-}). Recently, direct interactions of collapsin response mediator protein 2 (CRMP-2)—a protein involved in neurite outgrowth, guidance and axonogenesis—and neurofibromin have been demonstrated. It has been demonstrated that CRMP-2 associates with and influences the function of presynaptic Ca²⁺ channels involved in transmitter release. It was also shown that CRMP-2 influences axonal outgrowth and synaptic connectivity of neurons in the brain. These data indicate that a lack of neurofibromin in NF1 alters CRMP-2 function leading to impaired axonal connections and transmitter release which results in abnormal learning and cognitive function. Transmission regulatory peptides (TRAPs) that uncouple the interaction between neurofibromin and CRMP-2 have been identified. These TRAPs can be used to modulate the neurofibromin-CRMP-2 signaling cascade and to observe its effect on synaptic transmission in sensory neurons from wildtype and Nfl^{+/-} mice. Specifically, these molecules can be used to determine: (1) the exact site(s) of interactions between neurofibromin and CRMP-2 using a robotic high-throughput peptide tiling method combined with far-Westerns; (2) explore toxicity and in vitro uptake of various cell-penetration peptide conjugates (e.g., antennapedia, TAT, polyarginine) of these neurofibromin and CRMP-2 interaction disrupting peptides (i.e., the TRAPs); and (3) measure release of the peptide transmitter calcitonin gene related peptide (CGRP) from sensory neurons of wildtype and Nfl^{+/-} mice treated with these TRAPs conjugated to optimal cell-penetrating peptides. The effects of the TRAPs on voltage-gated ion channels (e.g., Na⁺ and Ca²⁺), neuronal excitability and behavior (e.g., the Morris water maze learning and memory test) can also be determined. Utilization of these TRAPs in wildtype and Nfl^{+/-} mice will provide early indications of the potential usefulness of these and similar molecules in the treatment of mammals including humans.

A first set of embodiments of the present disclosure includes at least one polypeptide that binds to at least one portion of a CRMP-2 protein, wherein the binding of the at least one polypeptide to the portion of the CRMP-2 protein disrupts the interaction between neurofibromin and CRMP-2 proteins.

A second set of embodiments of the present disclosure includes the first set of embodiments, wherein the at least one polypeptide may be derived from a portion of the C-terminus of neurofibromin.

A third set of embodiments of the present disclosure includes the first and second set of embodiments, wherein the at least one polypeptide comprises at least 80, 81, 82, 83, 84, 85, 86, 87, 88, or 89 percent identity to at least one polypeptide selected from the group consisting of: SEQ ID NO: 1, SEQ ID NO: 2, and SEQ ID NO: 3.

A fourth set of embodiments of the present disclosure includes any of the first to the third set of embodiments, wherein the at least one polypeptide comprises at least 90, 91, 92, 93, 94, 95, 96, 97, 98, or 99 percent identity to at least one polypeptide selected from the group consisting of: SEQ ID NO: 1, SEQ ID NO: 2, and SEQ ID NO: 3.

A fifth set of embodiments of the present disclosure includes any of the first to the fourth set of embodiments, wherein the at least one of the polypeptides is selected from the group consisting of: SEQ ID NO: 1, SEQ ID NO: 2, and SEQ ID NO: 3.

A sixth set of embodiments of the present disclosure includes any of the first to the fifth set of embodiments, wherein the at least one of the polypeptides comprises SEQ ID NO: 1.

A seventh set of embodiments of the present disclosure includes any of the first to the fifth set of embodiments, wherein the at least one of the polypeptides comprises SEQ ID NO: 2.

An eighth set of embodiments of the present disclosure includes any of the first to the fifth set of embodiments, wherein the at least one of the polypeptides comprises SEQ ID NO: 3.

A ninth set of embodiments of the present disclosure includes at least one polypeptide that binds to at least one portion of a CaV2.2 protein, wherein the binding of the at least one polypeptide to the portion of the CaV2.2 protein disrupts the interaction between CaV2.2 and CRMP-2 proteins.

A tenth set of embodiments of the present disclosure includes the ninth set of embodiments, wherein the at least one polypeptide may be derived from Ca²⁺ channel binding domains (CBDs) of a CRMP-2 protein.

An eleventh set of embodiments of the present disclosure includes the ninth and the tenth set of embodiments, wherein the at least one polypeptide comprises at least 80, 81, 82, 83, 84, 85, 86, 87, 88, 89, 90, 91, 92, 93, 94, 95, 96, 97, 98, or 99 percent identity to SEQ ID NO: 5.

A twelfth set of embodiments of the present disclosure includes any of the ninth and the eleventh set of embodiments, the at least one polypeptide comprises SEQ ID NO:5.

A thirteenth set of embodiments of the present disclosure includes a compound of the formula X—Z, wherein X is a polypeptide having at least 80, 81, 82, 83, 84, 85, 86, 87, 88, or 89 percent identity to at least one polypeptide selected from the group consisting of: SEQ ID NO: 1, SEQ ID NO: 2, SEQ ID NO: 3, and SEQ ID NO: 5, wherein Z is at least one polypeptide having at least 80, 81, 82, 83, 84, 85, 86, 87, 88, or 89 percent identity to at least one polypeptide selected from the group consisting of: SEQ ID NO: 4 and SEQ ID NO:6, and wherein X and Z are fused to one another.

A fourteenth set of embodiments of the present disclosure includes the thirteenth set of embodiments, wherein X is a polypeptide having at least 90, 91, 92, 93, 94, 95, 96, 97, 98, or 99 percent identity to at least one polypeptide selected from the group consisting of: SEQ ID NO: 1, SEQ ID NO: 2, SEQ ID NO: 3, and SEQ ID NO: 5.

A fifteenth set of embodiments of the present disclosure includes the thirteenth and the fourteenth set of embodiments, wherein Z is at least one polypeptide having at least 90, 91, 92, 93, 94, 95, 96, 97, 98, or 99 percent identity to at least one polypeptide selected from the group consisting of: SEQ ID NO: 4 and SEQ ID NO: 6.

A sixteenth set of embodiments of the present disclosure includes any of the thirteenth and the fifteenth set of embodiments, wherein X is a polypeptide comprising at least one polypeptide selected from the group consisting of: SEQ ID NO: 1, SEQ ID NO: 2, SEQ ID NO: 3, and SEQ ID NO: 5.

A seventeenth set of embodiments of the present disclosure includes any of the thirteenth and the sixteenth set of embodiments, wherein Z is at least one polypeptide comprising at least one polypeptide selected from the group consisting of: SEQ ID NO: 4 and SEQ ID NO: 6.

An eighteenth set of embodiments of the present disclosure includes any of the thirteenth and the seventeenth set of embodiments, wherein the X and Z are fused to one another via a peptide bond. In other embodiments, the X and Z are fused using a linker, wherein the linker comprises at least one amino acid.

A nineteenth set of embodiments of the present disclosure includes any of the thirteenth and the eighteenth set of embodiments, wherein X is a polypeptide comprising SEQ ID NO: 1.

A twentieth set of embodiments of the present disclosure includes any of the thirteenth and the eighteenth set of embodiments, wherein X is a polypeptide comprising SEQ ID NO: 2.

A twenty first set of embodiments of the present disclosure includes any of the thirteenth and the eighteenth set of embodiments, wherein X is a polypeptide comprising SEQ ID NO: 3.

A twenty second set of embodiments of the present disclosure includes any of the thirteenth and the eighteenth set of embodiments, wherein X is a polypeptide comprising SEQ ID NO: 5.

A twenty third set of embodiments of the present disclosure includes any of the thirteenth and the eighteenth set of embodiments, wherein the compound of the formula X—Z is one fusion peptide having at least 90, 91, 92, 93, 94, 95, 96, 97, 98, 99, or 100 percent identity to at least one fusion peptide selected from the group consisting of: SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 14.

A twenty fourth set of embodiments of the present disclosure includes a method of modulating synaptic transmissions, including the steps of providing at least one polypeptide that includes at least one of the polypeptide sequences according to any of the first to the twenty third set of embodiments, and contacting said at least one polypeptide with at least one protein selected from the group consisting of: CaV2.2 and CRMP-2.

A twenty fifth set of embodiments of the present disclosure includes a method of treating a patient, including the steps of providing at least one compound according to any of the first to the twenty third set of embodiments or a pharmaceutically acceptable salt thereof.

A twenty sixth set of embodiments of the present disclosure includes the twenty fifth set of embodiments, wherein said compound is formulated for administering to a patient.

A twenty seventh set of embodiments of the present disclosure includes any one of the twenty fifth and the twenty sixth set of embodiments, wherein the method further includes the step of administering at least one therapeutically effective dose of said compound to a patient.

A twenty eighth set of embodiments of the present disclosure includes any one of the twenty fifth and the twenty seventh set of embodiments, wherein the patient is a mammal or a human being.

A twenty ninth set of embodiments of the present disclosure includes any of the twenty fifth and the twenty eighth set of embodiments, wherein the patient is diagnosed with neurofibromatosis type 1 (NF1).

A thirtieth set of embodiments of the present disclosure includes a kit for treating a patient comprising at least one therapeutically effective dose of the at least one compound according to any of the first to the twenty fourth set of embodiments or a pharmaceutically acceptable salt thereof.

A thirty first set of embodiments of the present disclosure includes the thirtieth set of embodiments, wherein said compound in the kit is formulated for injection.

A thirty second set of embodiments of the present disclosure includes any of the thirtieth and the thirty first set of embodiments, wherein said compound in the kit is formulated with at least one additional material that helps to preserve the activity of said compound.

Some embodiments of the invention include peptides that interact with CRMP-2, comprising at least one of the peptides selected from the group consisting of SEQ ID NO: 1, SEQ ID NO: 2 and SEQ ID NO: 3. In some embodiments these peptides are individually fused to cell-membrane transduction domain of the human immunodeficiency virus-type 1 (HIV-1) (SEQ ID NO: 4 or SEQ ID NO:6).

Still other embodiments of the invention include methods of modulating synaptic transmissions, comprising the steps of: providing at least one peptide that includes at least one of the peptide sequences selected from the group consisting of SEQ ID NO: 1, SEQ ID NO: 2 and SEQ ID NO: 3; and contacting said at least one peptide sequence with CRMP-2. In some embodiments of the invention at least one of these sequences is fused to a portion of SEQ ID NO: 4. In some embodiments of the invention at least one of these sequences is fused to a portion of SEQ ID NO: 6.

### BRIEF DESCRIPTION OF THE SEQUENCES

SEQ ID NO: 1. RVSHGQIKQIIRILS, a peptide encompassing a portion of the C-terminus of neurofibromin.

SEQ ID NO: 2. KGYRHPSPAIVARTV, a peptide encompassing a portion of the C-terminus of neurofibromin.

SEQ ID NO: 3. YLQSFGFNGLWRFAG, a peptide encompassing a portion of the C-terminus of neurofibromin.

SEQ ID NO: 4. GRKKRRQRRRPQ, a portion of the cell-membrane transduction domain of the human immunodeficiency virus-type 1 (HIV-1) transactivator of transcription (TAT) protein.

SEQ ID NO: 5. DFVYKRIKARSRLAE, a peptide derived from a portion of the CBD3 region.

SEQ ID NO: 6. YGRKKRRQRRR, a TAT-Sequence.

SEQ ID NO: 7. YGRKKRRQRRRRVSHGQIKQIIRILS, a fusion peptide comprising a TAT and a portion of the C-terminus of neurofibromin.

SEQ ID NO: 8. YGRKKRRQRRRKGYRHPSPAIVARTV, a fusion peptide comprising a TAT and a portion of the C-terminus of neurofibromin.

SEQ ID NO: 9. YGRKKRRQRRRYLQSFGFNGLWRFAG, a fusion peptide comprising a TAT and a portion of the C-terminus of neurofibromin.

SEQ ID NO: 10. YGRKKRRQRRRDFVYKRIKARSRLAE, a fusion peptide comprising a TAT and a portion of the C-terminus of CBD3 region.

SEQ ID NO: 11. GRKKRRQRRRPQRVSHGQIKQIIRILS, a fusion peptide comprising a TAT and a portion of the C-terminus of neurofibromin.

SEQ ID NO: 12. GRKKRRQRRRPQKGYRHPSPAIVARTV, a fusion peptide comprising a TAT and a portion of the C-terminus of neurofibromin.

SEQ ID NO: 13. GRKKRRQRRRPQYLQSFGFNGLWRFAG, a fusion peptide comprising a TAT and a portion of the C-terminus of neurofibromin.

SEQ ID NO: 14. GRKKRRQRRRPQDFVYKRIKARSRLAE, a fusion peptide comprising a TAT and a portion of the C-terminus of CBD3 region.

### BRIEF DESCRIPTION OF THE FIGURES

FIG. 1. A cartoon summarizing some of the components of the NF1-CRMP-2-calcium signal transduction pathway.

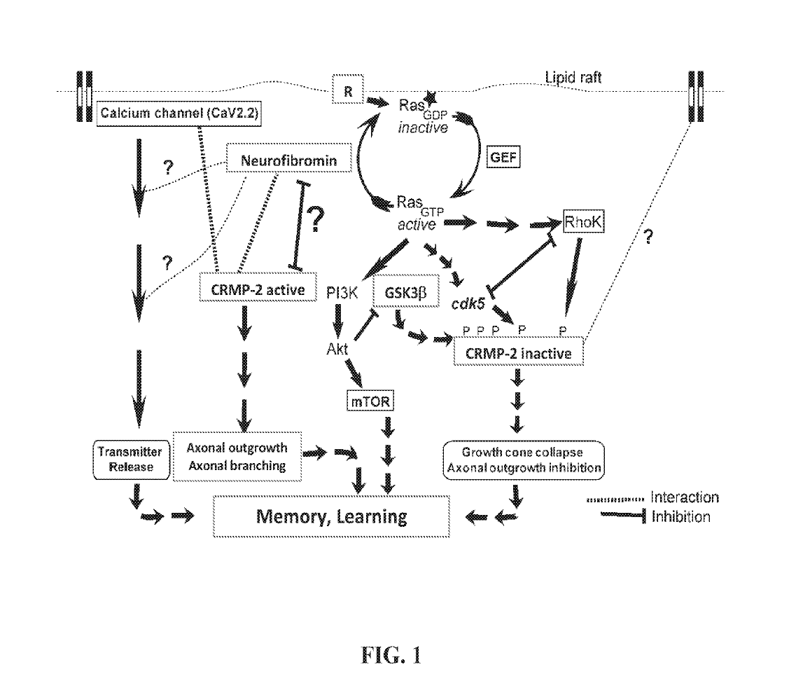

FIG. 2A. A bar graph illustrating the binding of polypeptides to CaV2.2 determined using SPOTS blot analysis.

FIG. 2B. A space filing model of the crystal structure of the CRMP-2 tetramer showing the position of the CBD3 peptide.

FIG. 2C. A graph showing example traces from neurons transfected with full-length CRMP-2 or CBD3 region of CRMP-2 at indicated voltage.

FIG. 2D. A bar graph illustrating the effect of normalized current density in CRMP-2 or CBD3 peptide expressing neurons.

FIG. 3A. A bar graph illustrating the effect of the TAT-CBD3 peptide on KCl-stimulated release of iCGRP.

FIG. 3B. A bar graph showing the total cellular iCGRP content measured in TAT or TAT-CBD3 expressing cells.

FIG. 4. A bar graph showing CRMP-2 binding to neurofibromin peptides.

### DETAILED DESCRIPTION

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and what it claims.

Unless specifically or implicitly stated otherwise the term ‘about’ as used herein means plus or minus 10 percent. For example, ‘about 1.0’ encompasses the range of 0.9 to 1.1.

A therapeutically effective amount is an amount of a biologically active compound that has a single or cumulative beneficial effect on the health or well being of a patient.

A mouse model of NF1 has been developed and it recapitulates many of the learning and memory defects observed in humans with NF1. With the aid of this mouse model it is now easier to identify key proteins involved in the signalling pathways affecting people with NF1. A new protein called CRMP-2, which is involved in growth and differentiation of neurons has been found to bind to NF1. By examining the interactions between NF1 and CRMP-2, it is possible to study how learning and memory are altered in the mouse model of NF1 and how common medications used to treat high cholesterol, such as statins, may repair this interaction. Ultimately, these studies will identify new targets for development of drugs and help to determine if any currently available drugs can quickly be used to treat at least some of the symptoms and/or consequences of NF1.

Recently, direct interactions of collapsin response mediator protein 2 (CRMP-2)—a protein involved in neurite outgrowth, guidance and axonogenesis—and neurofibromin have been demonstrated. In addition, it has been demonstrated that CRMP-2 associates with and may influence the function of presynaptic Ca²⁺ channels involved in transmitter release. It has also been demonstrated that CRMP-2 influences axonal outgrowth and synaptic connectivity of neurons in the brain. These results indicate that a lack of neurofibromin in NF1 alters CRMP-2 function leading to impaired axonal connections and transmitter release, which results in abnormal learning and cognitive function. The experiments disclosed herein can be used to readily identify transmission regulatory peptides (TRAPs) that uncouple the interaction between neurofibromin and CRMP-2. Once identified, these TRAPs can be used to study the neurofibromin-CRMP-2 signaling cascade and its effects on synaptic transmission in sensory neurons from wildtype and Nfl^{+/-} mice. Specifically, determination that can be made with these molecules include the following: (1) identifying the site(s) of interactions between neurofibromin and CRMP-2 using, for example, a robotic high-throughput peptide tiling method combined with far-Westerns; (2) exploring the toxicity and in vitro uptake of various cell-penetration peptide conjugates (e.g., antennapedia, TAT, polyarginine) of these neurofibromin and CRMP-2 interaction disrupting peptides (i.e., the TRAPs); and (3) measuring the release of the peptide transmitter calcitonin gene related peptide (CGRP) from sensory neurons of wildtype and Nfl^{+/-} mice treated with these TRAPs conjugated to optimal cell-penetrating peptides. Additional studies include testing the effects of the TRAPs on voltage-gated ion channels (e.g., Na⁺ and Ca²⁺), neuronal excitability and behavior (e.g., the Morris water maze learning and memory test). Robotic SPOTS blots have been used to identify a short peptide that not only uncouples the interaction between CRMP-2 and Ca²⁺ channels but also dramatically reduces transmitter release in sensory neurons from Nfl^{+/-} mice. Utilization of these TRAPs in wildtype and Nfl^{+/-} mice will provide early indications of the potential usefulness of these molecules in clinical trials in humans.

Some methods and compounds for suppressing pain by uncoupling collapsing response mediator protein 2 (CRMP-2) from the presynaptic calcium channel complex has been reported. See U.S. Pat. No. 9,018,173, the disclosure of which is incorporated by reference in its entirety to the extent it is not inconsistent with the explicit teachings of this specification

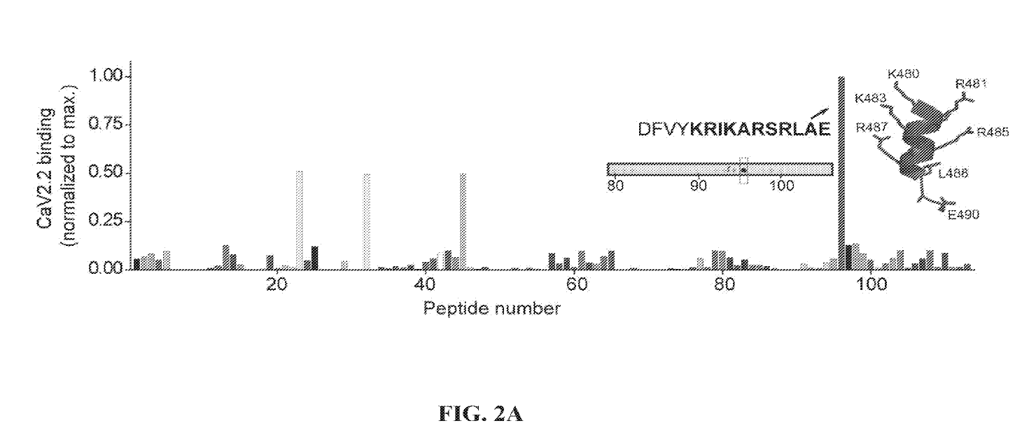

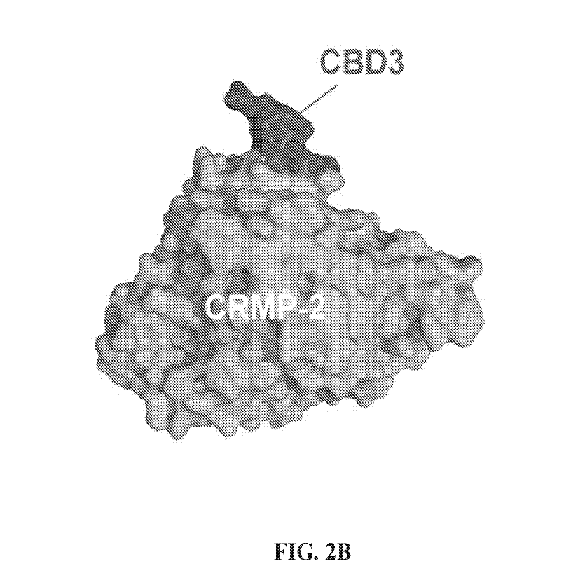

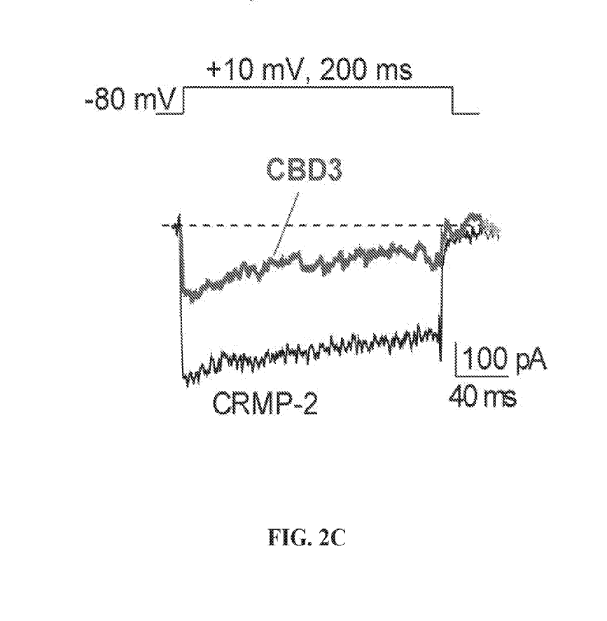

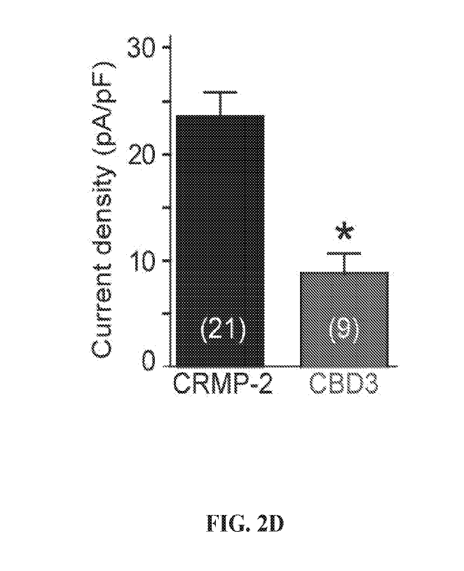

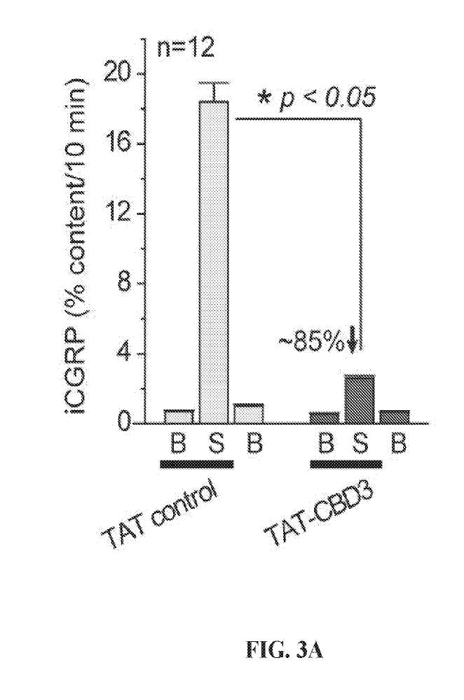

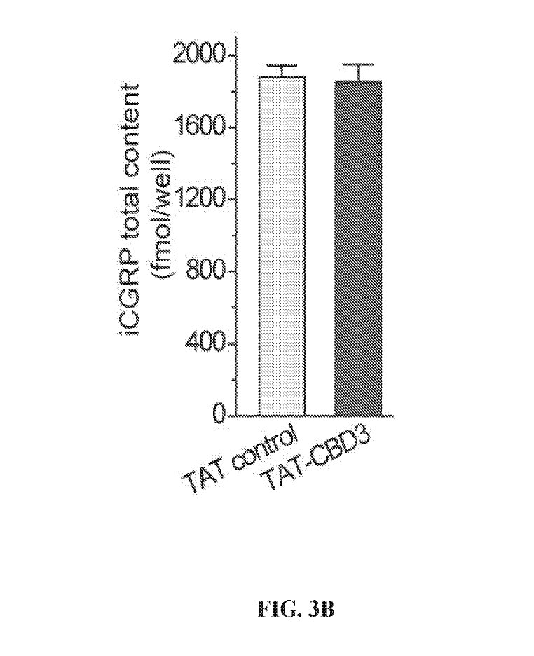

Referring now to FIGS. 2 and 3, a short polypeptide that disrupts the interaction between CRMP-2 and Ca²⁺ channels has been identified. Utilizing a robotic high-throughput peptide tiling method, a peptide library of 10-15 mers (with an overlap of 12 aa) spanning the entire length of CRMP-2 protein was synthesized using the InTavis Multipep RS SPOT synthesizer (InTavis, Germany); a total of 113 peptides were synthesized meeting these requirements. Then, using a far-Western approach (14, 15) the membrane harboring the CRMP-2 peptides was incubated overnight with enriched CaV2.2 protein purified from rat brain synaptosomes (16-18). CaV2.2 bound specifically to CRMP-2 polypeptides on the membrane was detected by blotting with a polyclonal CaV2.2 antibody. The intensity of each spot was plotted relative to the maximal binding observed. Spots blot analysis of CRMP-2 polypeptide binding to CaV2.2 revealed a high affinity peptide (sequence shown in 1 letter code). Three putative polypeptides derived from Ca²⁺ channel binding domains (CBDs) showed modest to strong binding (FIG. 2A). FIG. 2A also shows a partial structure of the CBD3 peptide (helix). Modest binding was also observed with another peptide (#45 in FIG. 2A); however this peptide was not tested further as this region is buried within the CRMP-2 structure. The relatively surface exposed nature of the CBD3 region (FIG. 2A) suggests that a peptide derived from this region could be useful in uncoupling the CaV2.2-CRMP-2 interaction and thus interfere with CaV2.2 trafficking and transmitter release.

Recently, it was shown that CRMP-2 facilitates CaV2.2 membrane trafficking in hippocampal and sensory neurons (12, 13), the hypothesis that the CBD3 peptide would act in a dominant negative manner to inhibit this CRMP-2 mediated trafficking was tested. Plasmids containing full-length CRMP-2 or the CBD3 peptide fused to enhanced green fluorescent protein (EGFP) were transiently transfected into human embryonic kidney 293 (HEK293) cells expressing CaV2.2. The majority of cells (90%) transfected with full-length CRMP-2 exhibited CaV2.2 surface staining. In contrast, only ˜10% of the CBD3 peptide transfected cells exhibited CaV2.2 staining on the surface, with most of the CaV2.2 trapped in clusters within the ER and Golgi (data not shown). Since CBD3 peptide expression was sufficient in preventing forward trafficking of CaV2.2, it is possible that this would result in loss of currents via these channels. To test this, neurons were transfected with full-length CRMP-2 or CBD3 peptide and measured CaV2.2 currents 2 days post-transfection using whole-cell voltage clamp electrophysiology. Expression of CBD3 reduced peak Ca²⁺ current density by ˜64% compared to CRMP-2 neurons (FIGS. 2C and 2D). These results are consistent with the hypothesis that CBD3 peptide acts in a dominant negative manner to suppress trafficking and surface expression of CaV2.2.

It has been reported that spinal cord slices as well as sensory neurons from Nfl^{+/-} mice exhibit an increased release of the neuropeptides, substance P (SP) and calcitonin gene-related peptide (CGRP), upon chemical stimulation (19). These studies are consistent with elegant work from the Silva laboratory which also showed increase transmitter release Nfl^{+/-} mice (1, 3). Therefore, to test if the CBD3 peptide could disrupt synaptic signaling in an NF1 context, the release of CGRP from sensory neurons of Nfl^{+/-} mice treated with TAT-conjugated versions of CBD3 or a scramble control was measured.

Referring now to FIG. 3A, CBD3 peptide reduces KCl-stimulated release of CGRP from DRGs of Nfl^{+/-} mice. Juvenile Nfl^{+/-} mice neurons were grown for 5-7 days prior to the release experiments. Bar graph of immunoreactive CGRP (iCGRP) release expressed as mean percent total iCGRP content of cells in each well±s.e.m. (n=12 wells/condition). Neuropeptide release was measured from cells treated with normal HEPES buffer containing 3.5 mM KCl (basal, B), HEPES buffer containing 50 mM KCl (S), and HEPES buffer containing 3.5 mM KCl again. Asterisks (*) indicate statistically significant differences in iCGRP release between TAT control and 10 μM TAT-CBD3 treatment groups using an ANOVA with Dunnett's post-hoc test (p<0.05). Unexpectedly, a dramatic 85% reduction in KCl-stimulated CGRP release was observed in dorsal root ganglion neurons from Nfl^{+/-} mice treated with TAT-CBD3 compared with TAT-control. The decrease in KCl-stimulated CGRP release observed in TAT-CBD3-treated neurons was not caused by a decrease in the total cellular content of iCGRP as there was no significant difference in neuropeptide content between the two conditions (FIG. 3B). Viability of cells treated with TAT peptides for as long as 12 hr was not affected by any of the treatments, ruling out the possibility that cell death may be contributing to the observed differences in transmitter release (data not shown).

The 10-12 mer TRAP peptide (typically 3000 molecular weight) identified from the neurofibromin-CRMP-2 robotic screening can be rendered cell-permeant by fusing each to the cell-membrane transduction domain of the human immunodeficiency virus-type 1 (HIV-1) transactivator of transcription (TAT) protein (20). Random or scramble peptides fused to TAT will serve as controls. A peptide outside the transduction domain of TAT will serve as an additional negative control. To ensure that the peptides are taken up the cells, the peptides will be conjugated to the fluorophore dansyl chloride. To test the peptides, they can be bath applied and then monitored for fluorescence. Fluorescence in the cytoplasm of the neurons would represent successful entry into cells whereas cultures treated with TAT-ntd (non transduction domain), devoid of the TAT transduction domain, would exhibit only background signal indicating no peptide uptake. CBD3 can also be synthesized with alternative cell-penetrating peptide conjugates such as antennapedia (AIP), transportan or polyarginine (21) as differences in the efficacy, toxicity, and uptake mechanisms of these cell-penetrating peptides can be critical factors in determining the effectiveness of the cargo.

The interaction of neurofibromin-CRMP-2 was partially mapped in order to identify peptides capable of blocking the modulatory effects of this protein complex on synaptic transmission. It is expected that transmission regulatory peptides (TRAPs) identified in wildtype and Nfl^{+/-} mice will prove to be useful in identifying molecules of clinical utility in various mammals including humans.

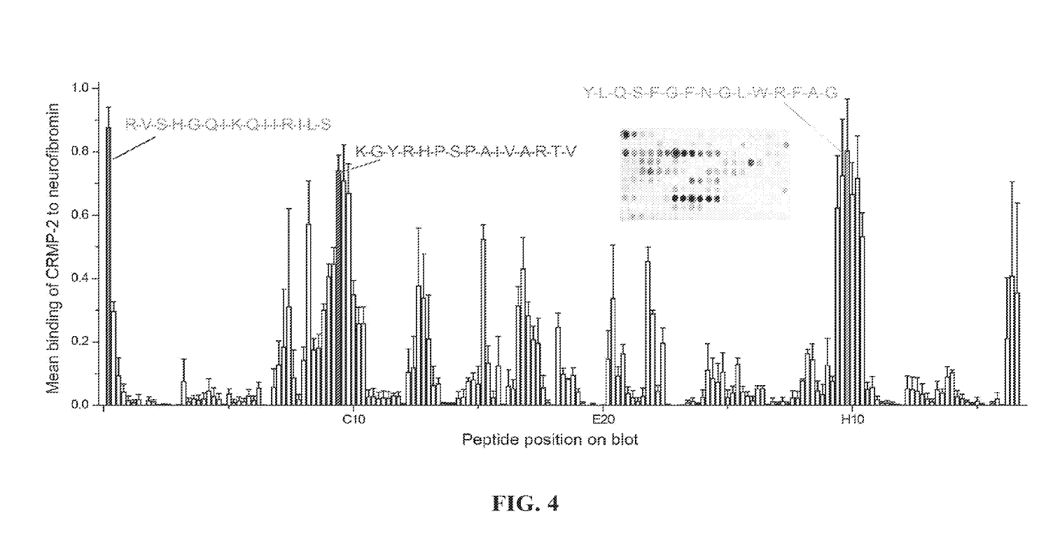

FIG. 4 presents plot of mean binding of CRMP-2 to neurofibromin versus peptide blot position. Neurofibromin peptides (15-mers) were spotted onto a SPOTS blot membrane from amino acids 2260-2818 of CRMP-2, a region where neurofibromin has been reported to bind CRMP-2. The blots were blocked, then overlaid with wild-type mouse brain lysate in NF1 solubilization buffer, washed briefly, then incubated with poly CRMP-2 (Sigma) or mono CRMP-2+poly CRMP-2 phospho Ser522 and Ser555 antibodies. The blots were scanned (Licor) and the spot intensity normalized to the maximum intensity in each series. The normalized data was then averaged and the mean+/−SEM is shown in the graph above (n=3).

Referring now to FIG. 4, peptides encompassing the C-terminus of neurofibromin were spotted with CRMP-2 and the resulting blot was probed with three different CRMP-2 antibodies. This resulted in the identification of three peaks that have now been synthesized. These peptides are: SEQ ID NO: 1, RVSHGQIKQIIRILS; SEQ ID NO: 2, KGYRHPSPAIVARTV, and SEQ ID NO: 3, YLQSFGFNGLWRFAG. In order to facilitate their entry into cells, each peptide may be fused to a portion of the cell-membrane transduction domain of the human immunodeficiency virus-type 1 (HIV-1) transactivator of transcription (TAT) protein (SEQ ID NO: 4 and/or SEQ ID NO: 6).

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

### REFERENCES

1. Costa R M and Silva A J (2002) Molecular and cellular mechanisms underlying the cognitive deficits associated with neurofibromatosis 1. J. Child Neurol. 17:622-626
2. Jacks T, Shih T S, Schmitt E M, Bronson R T, Bernards A and Weinberg R A (1994) Tumour predisposition in mice heterozygous for a targeted mutation in Nfl. Nat. Genet. 7:353-361
3. Cui Y, Costa R M, Murphy G G, Elgersma Y, Zhu Y, Gutmann D H, Parada L F, Mody I and Silva A J (2008) Neurofibromin regulation of ERK signaling modulates GABA release and learning. Cell. 135:549-560
4. Zhu Y, Romero M I, Ghosh P, Ye Z, Charnay P, Rushing E J, Marth J D and Parada L F (2001) Ablation of NF1 function in neurons induces abnormal development of cerebral cortex and reactive gliosis in the brain. Genes Dev. 15:859-876
5. Patrakitkomjorn S, Kobayashi D, Morikawa T, Wilson M M, Tsubota N, Irie A, Ozawa T, Aoki M, Arimura N, Kaibuchi K, Saya H and Araki N (2008) Neurofibromatosis type 1 (NF1) tumor suppressor, neurofibromin, regulates the neuronal differentiation of PC12 cells via its associating protein, CRMP-2. J. Biol. Chem. 283:9399-9413
6. Khanna R, Schmutzler B S, Brittain J M and Hingtgen C M (2009) Neurofibromin, CRMP-2 and presynaptic calcium channel proteins control synaptic transmission in a mouse model of neurofibromatosis type 1. pages 92-93. Children's Tumor Foundation Conference on Neurofibromatosis Jun. 13-16, 2009 (Portland, Oreg.)
7. Wang L H and Strittmatter S M (1996) A family of rat CRMP genes is differentially expressed in the nervous system. J. Neurosci. 16:6197-6207
8. Inagaki N, Chihara K, Arimura N, Menager C, Kawano Y, Matsuo N, Nishimura T, Amano M and Kaibuchi K (2001) CRMP-2 induces axons in cultured hippocampal neurons. Nat. Neurosci. 4:781-782
9. Nishimura T, Fukata Y, Kato K, Yamaguchi T, Matsuura Y, Kamiguchi H and Kaibuchi K (2003) CRMP-2 regulates polarized Numb-mediated endocytosis for axon growth. Nat. Cell Biol. 5:819-826
10. Arimura N, Menager C, Fukata Y and Kaibuchi K (2004) Role of CRMP-2 in neuronal polarity. J. Neurobiol. 58:34-47
11. Schmidt E F and Strittmatter S M (2007) The CRMP family of proteins and their role in Sema3A signaling. Adv. Exp. Med. Biol. 600:1-11.:1-11
12. Brittain J M, Piekarz A D, Wang Y, Kondo T, Cummins T R and Khanna R (2009) An atypical role for collapsin response mediator protein 2 (CRMP-2) in neurotransmitter release via interaction with presynaptic voltage-gated Ca2+ channels. J. Biol. Chem. 284:31375-31390
13. Chi X X, Schmutzler B S, Brittain J M, Hingtgen C M, Nicol G D and Khanna R (2009) Regulation of N-type voltage-gated calcium (CaV2.2) channels and transmitter release by collapsin response mediator protein-2 (CRMP-2) in sensory neurons. J. Cell Sci. 23:4351-4362
14. Wu Y, Li Q and Chen X Z (2007) Detecting protein-protein interactions by Far western blotting. Nat. Protoc. 2:3278-3284
15. Hall R A (2004) Studying protein-protein interactions via Blot overlay or Far Western Blot. In Protein-Protein Interactions, Methods and Application, Methods in Molecular Biology (Totowa, N.J.: Humana Press) pp. 167-174
16. Hell J W, Westenbroek R E, Warner C, Ahlijanian M K, Prystay W, Gilbert M M, Snutch T P and Catterall W A (1993) Identification and differential subcellular localization of the neuronal class C and class D L-type calcium channel alpha 1 subunits. J. Cell Biol. 123:949-962
17. Westenbroek R E, Hell J W, Warner C, Dubel S J, Snutch T P and Catterall W A (1992) Biochemical properties and subcellular distribution of an N-type calcium channel alpha 1 subunit. Neuron. 9:1099-1115
18. Sakamoto J and Campbell K P (1991) A monoclonal antibody to the beta subunit of the skeletal muscle dihydropyridine receptor immunoprecipitates the brain omega-conotoxin GVIA receptor. J. Biol. Chem. 266:18914-18919
19. Hingtgen C M, Roy S L and Clapp D W (2006) Stimulus-evoked release of neuropeptides is enhanced in sensory neurons from mice with a heterozygous mutation of the Nfl gene. Neuroscience. 137:637-645
20. Gupta B, Levchenko T S and Torchilin V P (2005) Intracellular delivery of large molecules and small particles by cell-penetrating proteins and peptides. Adv. Drug Deliv. Rev. 57:637-651
21. Jones S W, Christison R, Bundell K, Voyce C J, Brockbank S M, Newham P and Lindsay M A (2005) Characterisation of cell-penetrating peptide-mediated peptide delivery. Br. J. Pharmacol. 145:1093-1102

1. A compound, comprising at least one polypeptide that binds to at least one portion of a CRMP-2 protein, wherein the binding of the at least one polypeptide to the portion of the CRMP-2 protein disrupts the interaction between neurofibromin and CRMP-2 proteins.

2. The compound according to claim 1, wherein the at least one polypeptide is derived from a portion of the C-terminus of neurofibromin.

3. The compound according to claim 1, wherein the at least one polypeptide comprises at least 80 percent identity to at least one polypeptide selected from the group consisting of: SEQ ID NO: 1, SEQ ID NO: 2, and SEQ ID NO: 3.

4. (canceled)

5. The compound according to claim 1, wherein the at least one of the polypeptides is selected from the group consisting of SEQ ID NO: 1, SEQ ID NO: 2, and SEQ ID NO: 3.

6-8. (canceled)

9. A compound that uncouples CaV2.2 and CRMP-2, comprising:
at least one polypeptide that binds to at least one portion of a CaV2.2 protein, wherein the binding of the at least one polypeptide to the portion of the CaV2.2 protein disrupts the interaction between CaV2.2 and CRMP-2 proteins.

10. The compound according to claim 9, wherein the at least one polypeptide is derived from Ca²⁺ channel binding domains (CBDs) of a CRMP-2 protein.

11. The compound according to claim 9, the at least one polypeptide comprises at least 80 percent identity, at least 90 percent identity, or at least 95 percent identity to SEQ ID NO: 5.

12. The compound according to claim 9, the at least one polypeptide comprises SEQ ID NO:5.

13. A compound, comprising:
a compound of the formula X—Z;
wherein X is a polypeptide having at least 80 percent identity to at least one polypeptide selected from the group consisting of: SEQ ID NO: 1, SEQ ID NO: 2, SEQ ID NO: 3, and SEQ ID NO: 5; wherein Z is at least one polypeptide having at least 80 percent identity to at least one polypeptide selected from the group consisting of: SEQ ID NO: 4 and SEQ ID NO:6; and
wherein X and Z are fused to one another.

14. The compound according to claim 13, wherein X is a polypeptide having at least 90 percent identity to at least one polypeptide selected from the group consisting of: SEQ ID NO: 1, SEQ ID NO: 2, SEQ ID NO: 3, and SEQ ID NO: 5.

15. The compound according to claim 13, wherein Z is at least one polypeptide having at least 90 percent identity to at least one polypeptide selected from the group consisting of: SEQ ID NO: 4 and SEQ ID NO:6.

16. The compound according to claim 13, wherein X is a polypeptide comprising at least one polypeptide selected from the group consisting of: SEQ ID NO: 1, SEQ ID NO: 2, SEQ ID NO: 3, and SEQ ID NO: 5.

17. The compound according to claim 13, wherein Z is at least one polypeptide comprising at least one polypeptide selected from the group consisting of: SEQ ID NO: 4 and SEQ ID NO: 6.

18. The compound according to claim 13, wherein the X and Z are fused to one another via a peptide bond.

19-22. (canceled)

23. The compound according to claim 13, wherein the compound of the formula X—Z is one fusion peptide having at least 90 percent identity to at least one fusion peptide selected from the group consisting of: SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 14.

24. A method of modulating synaptic transmissions, comprising the steps of:
providing at least one polypeptide that includes at least one of the polypeptide sequences of claim 13; and
contacting said at least one polypeptide with at least one protein selected from the group consisting of: CaV2.2 and CRMP-2.

25. A method of modulating synaptic transmissions in a human patient, comprising the steps of:
administering to a human patient at least one compound according to claim 13, or a pharmaceutically acceptable salt thereof, in a therapeutically effective amount.

26-28. (canceled)

29. The method according to claim 25, wherein the human patient is diagnosed with neurofibromatosis type 1 (NF1).

30. A kit for treating a patient, comprising:
at least one therapeutically effective dose of the at least one compound according to claim 13, or a pharmaceutically acceptable salt thereof.

31. (canceled)

32. The kit according to claim 30, wherein said compound in the kit is formulated with at least one additional material that helps to preserve the activity of said compound.


SEQUENCE LISTING (14 sequences)

SEQ ID NO: 1 - protein, 15 aa, Artificial Sequence
  A synthesized peptide sequence
RVSHGQIKQIIRILS

SEQ ID NO: 2 - protein, 15 aa, Artificial Sequence
  A synthesized peptide sequence
KGYRHPSPAIVARTV

SEQ ID NO: 3 - protein, 15 aa, Artificial Sequence
  A synthesized peptide sequence
YLQSFGFNGLWRFAG

SEQ ID NO: 4 - protein, 12 aa, Artificial Sequence
  a modified TAT sequence
GRKKRRQRRRPQ

SEQ ID NO: 5 - protein, 15 aa, Artificial Sequence
  A synthesized peptide sequence
DFVYKRIKARSRLAE

SEQ ID NO: 6 - protein, 11 aa, Human immunodeficiency virus type 1
YGRKKRRQRRR

SEQ ID NO: 7 - protein, 26 aa, Artificial Sequence
  a synthesized peptide sequence
YGRKKRRQRRRRVSHGQIKQIIRILS

SEQ ID NO: 8 - protein, 26 aa, Artificial Sequence
  a fusion peptide
YGRKKRRQRRRKGYRHPSPAIVARTV

SEQ ID NO: 9 - protein, 26 aa, Artificial Sequence
  a fusion peptide
YGRKKRRQRRRYLQSFGFNGLWRFAG

SEQ ID NO: 10 - protein, 26 aa, Artificial Sequence
  fusion peptide
YGRKKRRQRRRDFVYKRIKARSRLAE

SEQ ID NO: 11 - protein, 27 aa, Artificial Sequence
  fusion peptide
GRKKRRQRRRPQRVSHGQIKQIIRILS

SEQ ID NO: 12 - protein, 27 aa, Artificial Sequence
  fusion peptide
GRKKRRQRRRPQKGYRHPSPAIVARTV

SEQ ID NO: 13 - protein, 27 aa, Artificial Sequence
  fusion peptide
GRKKRRQRRRPQYLQSFGFNGLWRFAG

SEQ ID NO: 14 - protein, 27 aa, Artificial Sequence
  fusion peptide
GRKKRRQRRRPQDFVYKRIKARSRLAE